SEQUENCE LISTING (19 sequences)

SEQ ID NO: 1 - DNA, 1770 bp, Solanum lycopersicum
  source 1..1770: mol_type = genomic DNA
  misc_feature 1..1770: note = CCA1 cDNA|Sl3_00 Solanum lycopersicum (S) wildtype
atgaaggctttgagtatagccgtagcagcattttcaacattccatacaactcgccctcgtctcttcactttcccaaacccttctacttcttcttctctccttcttccgtccttcaaactaaagagattttctatcttctccattcaacaaccgccgccggcgttagggttttacgccggagttacaacaatggtggagggctcagcatttcagccgttgtcagttcaagtgaaggaccatattgatttatcgccaaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccagtgcaatcccgatcagtcaaaacacttggaaacagcgaggatgcgccttttcgacgtatggattgattttgtcaacttaagagcagaagactacagtgagaacagtcgaattccaacaatgaaatttgggactgcaaaacaggatgctgatcgcagggatttaaccattaatagcttgttttacaacattaacaccagatcagtcgaagatcttacagaaagaggcattgcagatctgaagtctggaaaaattgtcactccactacctccaaagcagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgttacttgcttgggttcagcttgggaacttttaaaccttattggacgctcctcattttcagatgatcagaggagactttgcctgtatgctgctttatttttgccattcagagagaccatttatagagataacaaagccaaaaagattccagttgtcaattatatttttcgcaattctctcaagttgaaggccagtgatgcggaaatagtcatgagcttgcatacggtaactaggaaatttgtcaccttaatatcactactcatttctaaagacgatatccaagttcttgaagttgattggaaaagggataccattgaggttccaatagcttcaaaacttagaatattgactggtttgctacttcgagaaattaaagaattttggcgtgtcgctcttcttctatctatgctgctacatcctgttgacgttgtgtcctctacaagtttctcaaatgagaactttgaactggacaagagtagcggattatttaagacagtagagaatgcagtaagaacactaggtctggagaaggtttgggaaatgaaaccactggttaatggaaaggaaattatgaatattttggagattaaaagtggagggccagttgttagagaatggcaacaaaaactgctggagtggcagctcgcacacccttctggttcagctgaagaatgcatcgactggatgaagcaagcacagtcaaaacgtgcaagaaccgagtag

SEQ ID NO: 2 - protein, 589 aa, Solanum lycopersicum
  REGION 1..589: note = CCA1 protein|Wildtype CCA-adding enzyme
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVREWQQKLLEWQLAHPSGSAEECIDWMKQAQSKRARTE

SEQ ID NO: 3 - DNA, 1001 bp, Solanum lycopersicum
  source 1..1001: mol_type = genomic DNA
  misc_feature 1..1001: note = Promoter of the wildtype CCA1 gene
atatactatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatttaggtatgcctcaagttacgtgatttccaatagttttacaacaataataatacaaatataaatcattctttccattttttttgtctttctttctctcactttatacaaacacaaattgtgtaaaaaattatacaaaatttgtgtttgtataaaacgagagagagatttcatatacaaatattttaacttgattcaattgtatacgagtttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttatttttttttaaatatttatttttttttaaataaaaaaataaacttgccacatcatgaaagatgtagttgggatgacccgacccgttttctatgcttatgagagcgggtcatcacttgtccgttagaaagagagaagaa

SEQ ID NO: 4 - DNA, 10 bp, Solanum lycopersicum
  source 1..10: mol_type = genomic DNA
  misc_feature 1..10: note = Deletion in the promoter sequence
atatttattt

SEQ ID NO: 5 - DNA, 1101 bp, Solanum lycopersicum
  source 1..1101: mol_type = genomic DNA
  misc_feature 1..1101: note = CCA2 cDNA|Sl3_00 Solanum lycopersicum (S) wildtype
atgaaggctttgagtatagccgtagcagcattttcaacattccatacaactcgccctcgtctcttcactttcccaaacccttctacttcttcttctctccttcttccgtccttcaaactaaagagattttctatcttctccgttcaacaaacgccgccggcgttagggttttacgccggagttacaacaatggtggagggctcagcatttcagccgttgtcagttcaagtgaaggaccatattgatttatcgccgaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccaatgcaatcctgatcagtcaaaacacttggaaacagcaaggatgcgcctttccgacgtatggattgattttgtcaacttaagagcagaagactacagtgagaacagtcgaattccaacaatgaaatttggcactgcaaaacaggatgctgattgcagggatttaaccatcaatagcttgttttacaacattaacaccagatcagtcgaagatcttacagaaagaggcattgcagatctgaagtctggaaaaattgtcactccactacctccaaaacagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgtttcttgcttgggaacttttaaaccttattggacgctcctcattttcagatga

SEQ ID NO: 6 - DNA, 11 bp, Solanum lycopersicum
  misc_feature 1..11: note = Deletion in a CCA gene
  source 1..11: mol_type = genomic DNA
ttcagcttggg

SEQ ID NO: 7 - skipped

SEQ ID NO: 8 - protein, 589 aa, Solanum lycopersicum
  REGION 1..589: note = CCA1 protein|NCIMB43511, NCIMB43512
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVDGKEIMNILEIKSGGPVVSEWQQKLLEWQLAHPSGSAEECIDWMNQAQSKRARTE

SEQ ID NO: 9 - protein, 589 aa, Solanum lycopersicum
  REGION 1..589: note = CCA1 protein|TO1
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWERDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVSEWQQKLLEWQLAHPSGSAEECIDWMNQAQSKRARTE

SEQ ID NO: 10-11 - skipped (2 entries)

SEQ ID NO: 12 - DNA, 1000 bp, Solanum lycopersicum
  misc_feature 1..1000: note = CCA1 promotor|NCIMB43511, NCIMB43512
  source 1..1000: mol_type = genomic DNA
atggtatgaaatttaccatatacatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatataggtatgcctcaagttacgtgatttccaatagttttacaacaataataatacaaatataaatcattctttccatcttttttgtctttctttctctcactttatacaaacacaaattgtgtaaaaaattatacaaaatttgtgtttgtataaaacgagagagagatttcataaacaaatattttaacttgattcaattgtatacgaatttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttattttttttttaaataaaaaaataaacttgccacatcatgaaagatgtagttgggatgacccgacccgttttctatgcttatgagagcgggtcatcacttgtccgttagaaagagagaagaa

SEQ ID NO: 13 - DNA, 1001 bp, Solanum lycopersicum
  source 1..1001: mol_type = genomic DNA
  misc_feature 1..1001: note = CCA1 promotor|TO1
aatggtatgaaatttaccatatacaatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatataggtatgcctcaagttacgtgatttccaatagttttacaacaataataatacaaatataaatcattctttccatcttttttgtctttctttctctcactttatacaaacacaaattgtgtaaaaaattatacaaaatttgtgtttgtataaaacgagagagagatttcataaacaaatattttaacttgattcaattgtatacgaatttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttatttttttttaaataaaaaaataaacttgccacatcatgaaagatgtagttgggatgacccgacccgttttctatgcttatgagagcgggtcatcacttgtccgttagaaagagagaagaa

SEQ ID NO: 14-17 - skipped (4 entries)

SEQ ID NO: 18 - DNA, 17 bp, Solanum lycopersicum
  misc_feature 1..17: note = Deletion in the promoter sequence
  source 1..17: mol_type = genomic DNA
tttttaaatatttattt

SEQ ID NO: 19 - DNA, 17 bp, Solanum lycopersicum
  misc_feature 1..17: note = Deletion in the promoter sequence
  source 1..17: mol_type = genomic DNA
aaatatttatttttttt

(19) United States
(12) Patent Application Publication
(10) Pub. No.: US 2026/0185117 A1
(43) Pub. Date: Jul. 2, 2026

(54) CCA Gene For Virus Resistance

(71) Applicant: RIJK ZWAAN ZAADTEELT EN ZAADHANDEL B.V., De Lier (NL)

(72) Inventors: Jonathan KALISVAART, De Lier (NL); Raoul Jacobus Johannes Maria FRIJTERS, De Lier (NL); Daniël Johannes Wilhelmus LUDEKING, De Lier (NL); Alwin Johannes Marinus ROOVERS, De Lier (NL)

(21) Appl. No.: 19/530,841

(22) Filed: Feb. 5, 2026

Related U.S. Application Data
(63) Continuation-in-part of application No. 17/830,924, filed on Jun. 2, 2022, now abandoned.
Application No. 17/830,924 is a continuation-in-part of application No. PCT/EP2020/084504, filed on Dec. 3, 2020.

(30) Foreign Application Priority Data
Dec. 4, 2019 (WO) PCT/EP2019/083733

(51) Int. Cl. C12N 15/82; A01H 1/04; C12N 9/12; C12Q 1/6895

(52) U.S. Cl. CPC C12N 15/8283; A01H 1/045; C12N 9/1241; C12Q 1/6895; C12Q 2600/13; C12Q 2600/156; C12Y 207/07056

(57) ABSTRACT

The present invention relates to a modified CCA gene which encodes a CCA-adding enzyme, which modified CCA gene leads to resistance against a positive-strand RNA virus having a transfer RNA-like structure (TLS). The invention further relates to plants and seeds comprising the modified genes, methods for making and identifying such plants and use of the gene.

### RELATED APPLICATIONS AND INCORPORATION BY REFERENCE

This application is a continuation-in-part application of U.S. patent application Ser. No. 17/830,924, filed Jun. 2, 2022, which is a continuation-in-part application of international patent application Serial No. PCT/EP2020/084504 filed 3 Dec. 2020, which published as PCT Publication No. WO 2021/110855 on 10 Jun. 2021, which claims benefit of international patent application Serial No. PCT/EP2019/083733 filed 4 Dec. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically in XML format and is hereby incorporated by reference in its entirety. Said XML copy, created on Feb. 4, 2026, is named Y7954-01524_SL.xml, and is 25,321 bytes in size.

### FIELD OF THE INVENTION

The present invention relates to a modified gene which leads to resistance against a positive-strand RNA virus having a TLS. The invention further relates to a plant which may comprise said modified gene, methods for producing such a plant, and methods for identification of the modified gene and selection of such a plant. The invention also relates to a marker for identification of the modified gene in a plant, and to use of said marker.

### BACKGROUND OF THE INVENTION

Viral diseases pose one of the major threats growers have to deal with, both in protected and open field crop cultivation. Once a crop is infected, spread of the virus can occur rapidly through hard-to-control vectors, usually insects. In addition, cultivation methods often contribute to a further spread of the virus, by sap transmission through tools and fieldworkers.

Plant viruses typically depend on their hosts for rapid replication and spread, thereby infecting that same host with the disease. Different viruses have different systems which they deploy to achieve this goal. A certain group of viruses belonging to the positive-strand RNA viruses appears to use transfer RNA-like structures (TLSs) at their 3′-terminal genome sequence as an essential factor in these processes. These viral TLSs are capable of specific aminoacylation related to the order of the anticodon sequence that is present in their TLS structure, thereby mimicking universally present transfer RNA (tRNA) behaviour. Usually however, the TLSs of these viral genomes lack the CCA tail that is an essential tRNA property for the binding of an associated amino acid. Instead, the viral genome often terminates in 3′-CC; this CC-tail can however be adenylated through utilization of the tRNA nucleotidyltransferase, also called the ‘CCA-adding enzyme’, of any host plant in which the virus has entered. This adenylation, and subsequent aminoacylation, of the viral genome are thought to form an essential step in virus infection and spread, since they are recognized to play an important role in virus stabilization, translation, and replication. Several positive-strand RNA viruses belonging to the genera Tobamovirus, Tymovirus, or Bromovirus are examples that use this tRNA-mimicking system. Besides possessing a TLS at the 3′-end of their genome, these viruses generally also have a TLS at the 3′-ends of their sgRNA transcripts, which transcript TLSs could also have a function in the interaction with a CCA-adding enzyme of the host.

Numerous genes have been recognized for their involvement in virus resistance in plants. Virus resistance can be based on various mechanisms, and many different phases of plant development and plant defense pathways can be involved. However, for a large number of viruses no resistance gene has been identified yet. Especially for relatively new viruses, or viruses that are similar to others but break known resistances, there is always the challenge to identify a new source of resistance before the virus damage becomes too extensive. Newly identified resistance genes can also be an addition to the protection of crops against already known viral diseases.

Citation or identification of any document in this application is not an admission that such document is available as prior art to the present invention.

### SUMMARY OF THE INVENTION

It is an object of the present invention to provide a modified gene that leads to resistance against a positive-strand RNA virus that has a 3′-terminal transfer RNA-like structure (TLS).

The present invention provides a modified CCA gene which encodes a CCA-adding enzyme, which modified CCA gene leads to resistance against a positive-strand RNA virus having a TLS, wherein the modified CCA gene is selected from the group consisting of:
a gene which may comprise a nucleotide sequence that encodes a CCA-adding enzyme according to SEQ ID NO: 8;
a gene which may comprise a promoter sequence which may comprise SEQ ID NO: 12;
a gene which may comprise a nucleotide sequence that encodes a CCA-adding enzyme having a deletion, a substitution, or an insertion of at least one amino acid when compared to SEQ ID NO: 2;
a gene which may comprise a nucleotide sequence that encodes a CCA-adding enzyme having a deletion, a substitution, or an insertion of at least one amino acid when compared to SEQ ID NO: 8;
a gene which may comprise a nucleotide sequence that encodes a CCA-adding enzyme having at least 80% sequence identity to SEQ ID NO: 8; and
a gene which may comprise a promoter sequence having at least 80% sequence identity to SEQ ID NO: 12.

The invention further relates to plants and seeds which may comprise the modified genes, methods for making and identifying such plants and use of the gene.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposits

Seed of tomato Solanum lycopersicum comprising a modified CCA gene of the invention was deposited with NCIMB Ltd, Ferguson Building, Craibstone Estate, Bucksburn, Aberdeen AB21 9YA, UK on 7 Nov. 2019, under deposit accession numbers NCIMB 43511 and NCIMB 43512.

The Deposits with NCIMB Ltd, Ferguson Building, Craibstone Estate, Bucksburn, Aberdeen AB21 9YA, UK, under deposit accession numbers NCIMB 43511 and NCIMB 43512 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1—CDS sequence of SEQ ID NO: 1 (the wildtype CCA1 gene of Solanum lycopersicum).

FIG. 2—protein sequence of SEQ ID NO: 2 (the wildtype CCA-adding enzyme encoded by SEQ ID NO: 1), and SEQ ID NO: 8 (CCA-adding enzymes with modifications that lead to resistance). CCA1_NCIMB 43511 and CCA1_NCIMB 43512 are the same as SEQ ID NO: 8. CCA1_Ramyle F1 and CCA1_S13_00 are the same as SEQ ID NO: 2.

FIG. 3A—promoter sequences of SEQ ID NO: 3 (promoter of the wildtype CCA1 gene of Solanum lycopersicum) and SEQ ID NOS: 12. CCA1_NCIMB43511 43512 is the same sequence as SEQ ID NO: 12.

FIG. 3B shows an alternative alignment of a stretch before nucleotide 917, which stretch comprises a deletion in all of these sequences when compared to SEQ ID NO: 3.

FIG. 4—representation of the domains of the CCA-adding enzyme.

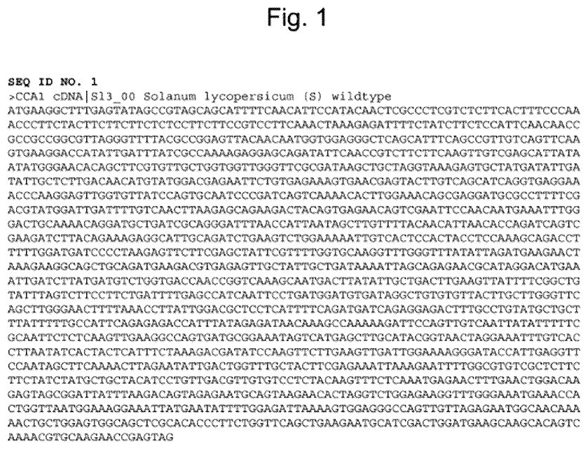

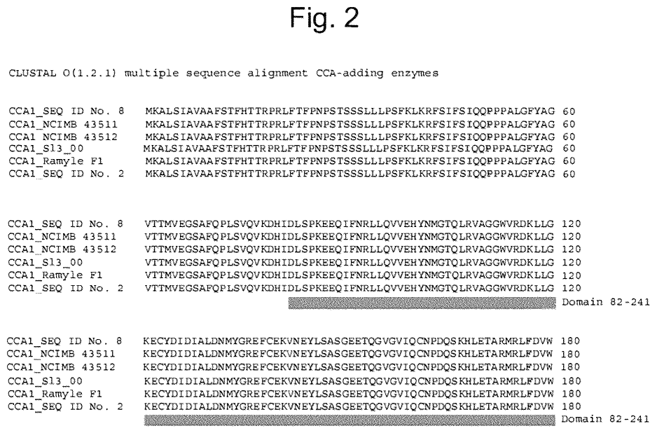

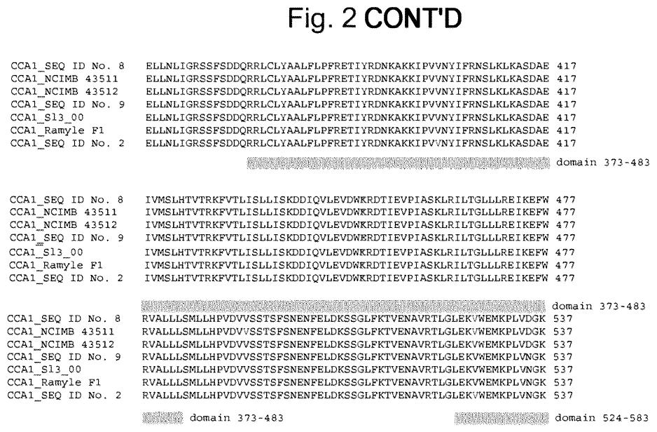

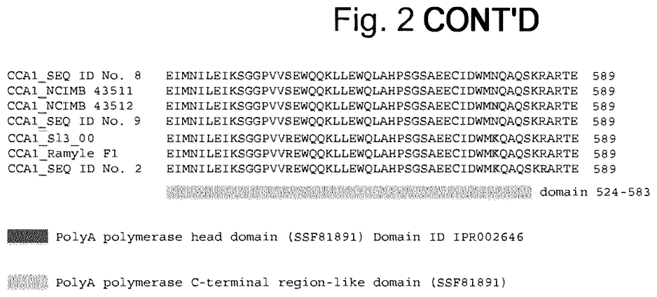

### DETAILED DESCRIPTION OF THE INVENTION

As used herein, a CCA gene is a gene encoding a CCA-adding enzyme. As used herein, a CCA gene is a gene which may comprise a wildtype CDS sequence represented by SEQ ID NO: 1, or a homologous gene which may comprise a sequence having at least 80% sequence identity to SEQ ID NO: 1; or a gene encoding a CCA-adding enzyme which may comprise SEQ ID NO: 2; or a gene encoding a homologous CCA-adding enzyme which may comprise a sequence having at least 80% sequence identity to SEQ ID NO: 2. As used herein, a gene also may comprise the 5′-UTR sequence, the promoter, and the 3′-UTR sequence of that gene.

The promoter of a CCA gene may comprise SEQ ID NO: 3, or may comprise a sequence having at least 80% sequence identity to SEQ ID NO: 3, preferably 85%, 90%, 93%, 95%, 96%, 97%, 98%, or 99%. A homologous CCA gene may comprise a sequence having at least 80% sequence identity to SEQ ID NO: 1, preferably 85%, 90%, 93%, 95%, 96%, 97%, 98%, or 99%. A homologous CCA-adding enzyme may comprise a sequence having at least 80% sequence identity to SEQ ID NO: 2, preferably 85%, 90%, 93%, 95%, 96%, 97%, 98%, or 99%.

A CCA-adding enzyme having at least 80% sequence identity to SEQ ID NO: 8 preferably may comprise at least one of a N535D substitution, an R553S substitution, or a K579N substitution. Any of those substitutions is alternatively a substitution on the corresponding position of a homologous sequence.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment.

The CCA-adding enzyme is active in most living organisms, and plays a crucial role therein, as it is essential for adding a CCA-tail to the 3′-end of the universally present transfer RNAs (tRNAs). In nearly all eukaryotes this CCA-tail, which is a prerequisite for aminoacylation of the tRNA, is not encoded by the tRNA gene, and it therefore has to be added post-transcriptionally. The specialized CCA-adding enzyme recognizes all tRNAs and is responsible for synthesis of a proper CCA-tail in all of them. Most eukaryotic genomes have only a single copy of a CCA gene that encodes the essential and highly conserved CCA-adding enzyme.

The CCA-adding enzyme is also involved in other RNA-related processes. One of its tasks is for example tRNA quality control, whereby the enzyme plays a role in tRNA repair, as well as in degradation of unstable or otherwise deviating tRNAs. By adding a double instead of a single CCA tail to RNA that is for some reason identified to be faulty, it tags this RNA for degradation. The CCA-adding enzyme is further also involved in processing of other non-coding RNAs, such as lncRNAs.

Because of the essential role of the CCA-adding enzyme, mutations in a CCA gene, especially mutations that are present in the highly conserved parts of the gene sequence, are anticipated to have a strong negative impact on the growth and development of a plant. Therefore, even though it was known that many viruses have a 3′-terminal transfer RNA-like structure (TLS) that makes use of the CCA-adding enzyme of the host plant for infection of that same host plant, the essential function made CCA genes an unlikely target in an approach to obtain virus resistance.

The present invention however presents a modification in a CCA gene that leads to virus resistance in a plant.

The modification in a CCA gene that leads to resistance against a positive-strand RNA virus having a TLS is a modification that is selected from the group consisting of:
a modification in the promoter sequence of a CCA gene;
a modification in the genomic sequence of a CCA gene;
a modification in the coding sequence (CDS) of a CCA gene;
a modification in a regulatory sequence of a CCA gene; and
a modification in a conserved domain of a CCA gene, or any combination thereof.

The modification in a CCA gene that leads to resistance will change the expression of said gene. Alternatively, or as a result, the modification can affect the activity and/or function of the encoded protein, or no protein can be encoded. The modification in the CCA gene of the invention may comprise a modification resulting in an amino acid change, a modification resulting in an early stop codon, a modification resulting in a truncated protein, or a modification resulting in a frameshift. Due to the modification the encoded protein has a changed function, a reduced function, or it is non-functional.

The changed expression of the CCA gene of the invention may comprise reduced expression, no expression, or silencing. The modification in the CCA gene of the invention may comprise a deletion, a substitution, or an insertion of at least one nucleotide in the nucleotide sequence of SEQ ID NO: 1 or a homologous sequence thereof, or of at least one amino acid in the encoded protein which may comprise SEQ ID NO: 2 or a homologous sequence thereof. The modification may comprise a modification that affects a conserved domain, such as an active site or catalytic domain, of the encoded protein, which is the CCA-adding enzyme.

In one embodiment, a modification that leads to resistance against a positive-strand RNA virus having a TLS may comprise a deletion in the promoter sequence of the CCA gene. The promoter of a CCA gene that is suitable to be modified to result in resistance may comprise a sequence having in order of increased preference at least 80%, 85%, 90%, 95%, 98%, 99%, or 100% sequence identity to SEQ ID NO: 3, provided that the promoter sequence may comprise SEQ ID NO: 4. A deletion in the promoter sequence of a CCA gene resulting in changed expression of said gene, and thereby in resistance, may comprise a deletion in a regulatory sequence, in particular a deletion in the TATA box, or a deletion which may comprise SEQ ID NO: 4 (Table 1).

In a preferred embodiment the deletion which may comprise SEQ ID NO: 4 is a deletion which may comprise SEQ ID NO: 18, or a deletion which may comprise SEQ ID NO: 19.

In one embodiment, the modification that leads to resistance against a positive-strand RNA virus having a TLS may comprise a SNP in the CDS of a CCA gene leading to an amino acid substitution. Optionally, the SNP leads to an amino acid substitution in a conserved domain, such as an active site or catalytic domain, of the CCA-adding enzyme. A conserved domain of the CCA-adding enzyme may comprise the PolyA_pol_head_domain (domain ID IPR002646, accessible on-line at the InterPro database) which may comprise positions 82 to 241 of SEQ ID NO: 2, or the corresponding positions in a homologous sequence having at least 80% sequence identity to SEQ ID NO: 2. A conserved domain also may comprise the polyA_pol_C-terminal region-like domain (domain ID SSF81891, accessible on-line at the Superfamily database). This domain may comprise three active sites and is positioned from amino acid 244 up to amino acid 583 of the CCA-adding enzyme which may comprise SEQ ID NO: 2, or at the corresponding positions of a homologous CCA-adding enzyme sequence having at least 80% sequence identity to SEQ ID NO: 2.

It was surprisingly found that the species Solanum lycopersicum diverges from the general rule and may comprise two CCA genes in its genome, which genes are highly homologous and share 95% sequence identity. The first CCA gene, identified herein as SICCA1, is represented by SEQ ID NO: 1. The second CCA gene in S. lycopersicum, identified herein as SICCA2, has an 11 bp deletion when compared to SICCA1, which deletion results in a frameshift and thereby in an early stop codon. The SICCA2 gene of S. lycopersicum is represented by SEQ ID NO: 5. The deletion in the SICCA2 gene as compared to the SICCA1 gene is present in exon 9 of the gene, and leads to an early stop codon in exon 10 of SICCA2. The deletion is specifically an 11 bp deletion corresponding to positions 1062 to 1072 of SEQ ID NO: 1. The deletion is in particular a deletion which may comprise SEQ ID NO: 6 (Table 1).

TABLE 1
Deletions in CCA genes.

| | |
|---|---|
| SEQ ID NO: 4 | ATATTTATTT |
| SEQ ID NO: 6 | TTCAGCTTGGG |
| SEQ ID NO: 18 | TTTTTAAATATTTATTT |
| SEQ ID NO: 19 | AAATATTTATTTTTTTT |

Tomato Brown Rugose Fruit Virus (ToBRFV) was first described by Luria et al ((2017): A new Israeli tobamovirus isolate infects tomato plants harboring Tm-22 resistance genes. PLOS ONE 12 (1): e0170429. Doi: 10.1371/journal.pone.0170429). At the time of that publication Tomato Brown Rugose Fruit Virus was still abbreviated as TBRFV, but in the meantime the commonly used abbreviation for this virus is ToBRFV, which is therefore now also used in the present application.

During even further research, several SICCA-gene polymorphisms were identified that result in ToBRFV resistance. Certain modifications were found in the CCA genes of wild tomato species, in particular in Solanum pimpinellifolium species; when these modifications were transferred to a ToBRFV susceptible S. lycopersicum plant, the S. lycopersicum plant became resistant to ToBRFV. A SNP resulting in an amino acid change that leads to resistance may comprise an A to T SNP on position 948 (A948T) of SEQ ID NO: 1, a C to T SNP on position 950 (C950T) of SEQ ID NO: 1, an A to G SNP on position 1348 (A1348G) of SEQ ID NO: 1, an A to G SNP on position 1603 (A1603G) of SEQ ID NO: 1, an A to T SNP on position 1659 (A1659T) of SEQ ID NO: 1, or a G to T SNP on position 1737 (G1737T) of SEQ ID NO: 1, or on any of the corresponding positions in a homologous sequence of SEQ ID NO: 1. Said nucleotide changes respectively result in a K316N substitution, an A317V substitution, an K450E substitution, an N535D substitution, an R553S substitution, or a K579N substitution in SEQ ID NO: 2, or an amino acid substitution at the corresponding positions of a homologous sequence of SEQ ID NO: 2.

As used herein, a X000Y mutation, SNP, or substitution means that the wildtype sequence has a nucleotide or amino acid X on position 000, which is changed to nucleotide or amino acid Y in the modified sequence.

SEQ ID NO: 8 may comprise an N535D mutation, an R553S mutation, and a K579N mutation.

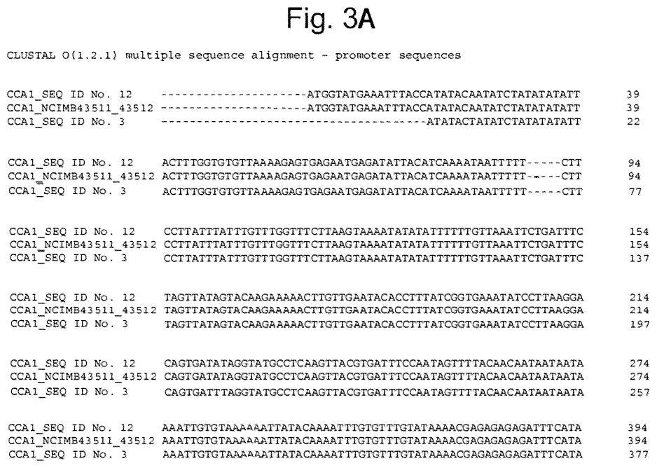

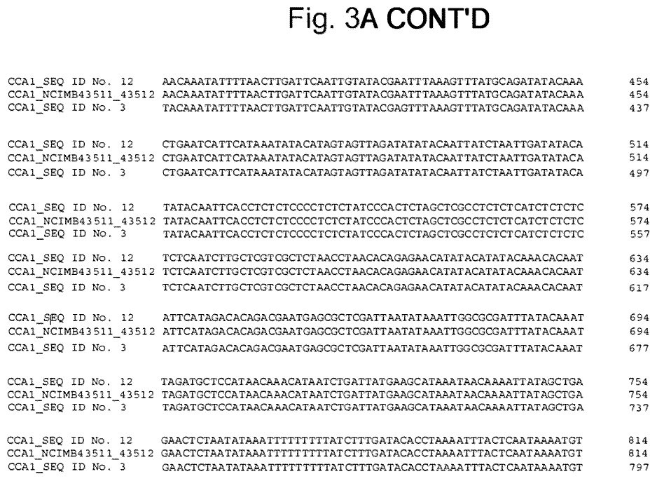

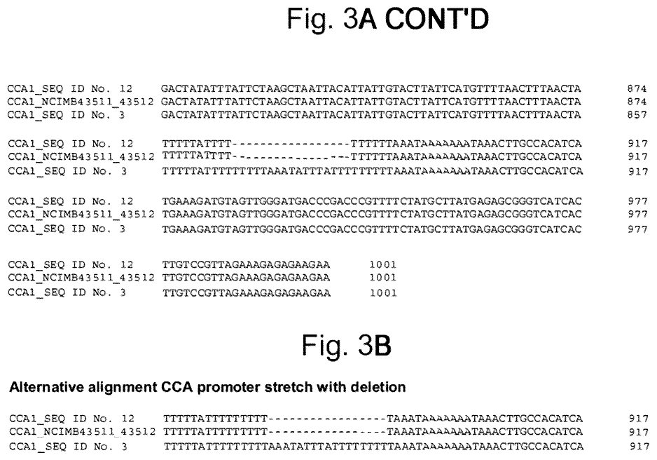

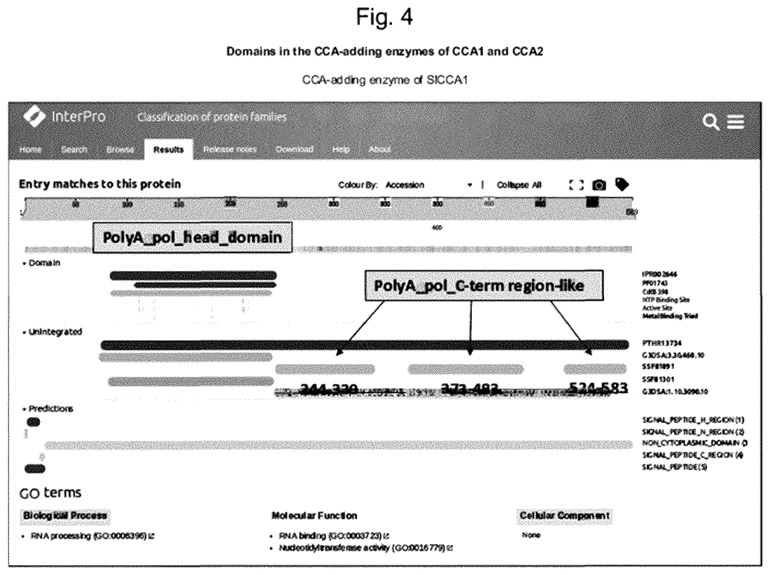

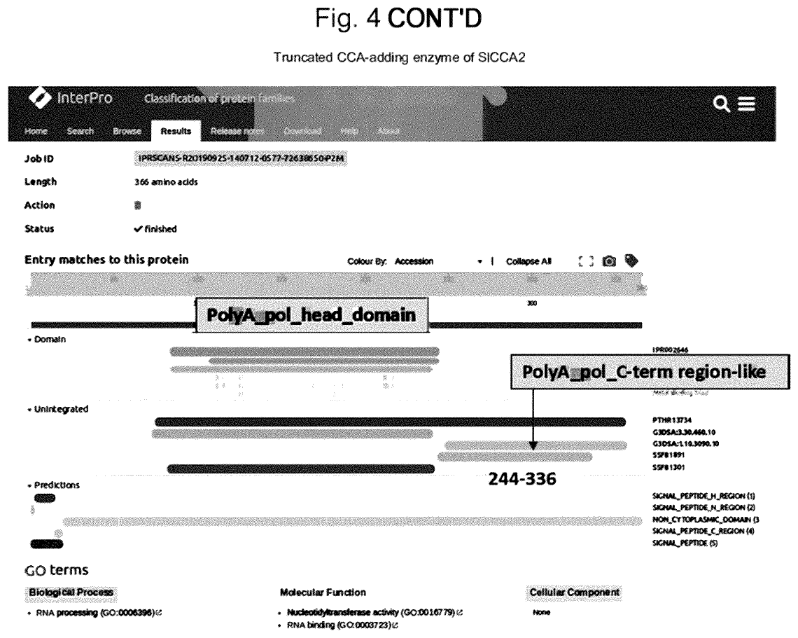

In addition, other polymorphisms that correlated with ToBRFV resistance in S. lycopersicum were found in the promoters of the CCA genes. A CCA1 gene that showed resistance had a deletion which may comprise SEQ ID NO: 4 in the promoter sequence when compared to the wildtype SEQ ID NO: 3. Other polymorphisms comprised nucleotide substitutions within the promoter sequence, as for example presented in the promoter sequences alignment of FIG. 3.

In one embodiment, the promoter of a modified CCA gene of the invention may comprise SEQ ID NO: 12. All of these promoter sequences have a deletion which may comprise SEQ ID NO: 4 when compared to the wildtype promoter sequence which may comprise SEQ ID NO: 3 (FIG. 3B).

As used herein, resistance against a positive-strand RNA virus having a TLS, in particular resistance against a Tobamovirus, more in particular resistance against ToBRFV, may comprise tolerance and/or field tolerance to the virus. Virus resistance can express itself on different levels, whereby different mechanisms are involved. When a plant is truly resistant to a virus, the infection and/or replication of the virus in the hostplant is restricted by the resistance mechanism. When a young plant bio-assay is performed, the resistant plant does not show susceptibility symptoms.

As used herein, when a plant is tolerant to a virus, virus replication and multiplication can take place in the plant, which can for example be measured through a qPCR experiment. Some mild symptoms can be observed in a bio-assay, but the impact of the presence of the virus on the fitness of the plant is strongly reduced as compared to the impact on a susceptible plant.

A specific form of tolerance is field tolerance, as used herein, when a plant is field tolerant, the host plant is not able to limit virus replication and multiplication, and the plants will show symptoms in a bio-assay performed under controlled conditions on young plants. However, when such a plant is grown in the field under normal cultivation practices, the host is able to reduce the impact of the virus presence on the plant's fitness, and no or limited symptoms will be seen. In addition, the yield of the crop will not be significantly reduced and will be comparable to the yield of a crop without the virus.

In one embodiment, a modification that leads to resistance against a positive-strand RNA virus having a TLS may comprise a combination of two or more of above-described modifications in one CCA gene, which combination can be modifications in the coding sequence, modifications in the promoter sequence, or a modification in the promoter sequence and a modification in the coding sequence. The modification can also be a combination of at least one modification in each of two CCA genes when two CCA genes are present in the genome of a plant, wherein the modifications in both CCA genes can be different or can be the same. The modifications can in particular be a combination of at least one modification in the gene represented by SEQ ID NO: 1; or a combination of at least one modification in the gene represented by SEQ ID NO: 1 and at least one modification in the promoter represented by SEQ ID NO: 3; or modifications in homologous sequences of SEQ ID NO: 1 and SEQ ID NO: 3.

A positive-strand RNA virus having a TLS may comprise a virus of the genus Tobamovirus, the genus Tymovirus, or the genus Bromovirus. A positive-strand RNA virus having a TLS is preferably a virus of the genus Tobamovirus, in particular a virus of the species ToBRFV or TMV or ToMV or CGMMV. The modification in the CCA gene of the invention preferably leads to ToBRFV resistance, optionally in combination with resistance against another virus, in particular another Tobamovirus.

The present invention relates to a plant which may comprise a modified CCA gene of the invention. The plant which may comprise the modified CCA gene is preferably a plant of the Solanaceae family, which may comprise a plant of the species Solanum lycopersicum, Capsicum annuum, Solanum melongena, Capsicum frutescens, Solanum tuberosum, Petunia spp, or Nicotiana tabacum. A plant of the invention is preferably a cultivated plant which is non-wild and has agronomical value, and is in particular agronomically elite. “Agronomically elite” means that a plant or a line has desirable agronomic characteristics such as high yield, maturity, disease resistance, and the like such that the plant or the line is appropriate for commercial cultivation.

In one embodiment, the plant which may comprise the modified CCA gene of the invention is resistant to a positive-strand RNA virus having a TLS, in particular to a virus of the genus Tobamovirus, the genus Tymovirus, or the genus Bromovirus, preferably a virus of the genus Tobamovirus. The positive-strand RNA virus is most preferably of the species Tomato Brown Rugose Fruit Virus (ToBRFV) or the species Tobacco Mosaic Virus (TMV) or the species Tomato Mosaic Virus (ToMV).

In a preferred embodiment, the plant of the invention is a plant of the species Solanum lycopersicum which may comprise a modified CCA gene, which plant is resistant to a Tobamovirus, in particular to Tomato Brown Rugose Fruit Virus (ToBRFV). The modification in a CCA gene in the S. lycopersicum plant of the invention may comprise a modification selected from the group which may comprise an A to T SNP on position 948 of SEQ ID NO: 1; a C to T SNP on position 950 of SEQ ID NO: 1; an A to G SNP on position 1348 of SEQ ID NO: 1; an A to G SNP on position 1603 of SEQ ID NO: 1; an A to T SNP on position 1659 of SEQ ID NO: 1; a G to T SNP on position 1737 of SEQ ID NO: 1; a deletion in the promoter of the CCA gene, in particular a deletion which may comprise SEQ ID NO: 4 from the promoter sequence which may comprise SEQ ID NO: 3; or corresponding modifications in homologous sequences of SEQ ID NO: 1 and SEQ ID NO: 3. A certain modification in a CCA gene can result in resistance of one or more categories of resistance.

An overview of SNP modifications in a CCA gene resulting in amino acid substitutions in its encoded protein, which is the CCA-adding enzyme, that form part of the present invention is presented in Table 4. The modification is indicated from susceptible (before the indicated position) to resistant (after the indicated position).

In one embodiment, a S. lycopersicum plant of the invention may comprise a CCA1 gene which may comprise SEQ ID NO: 8 and SEQ ID NO: 12.

ToBRFV resistance is determined by comparison to a control variety known to be ToBRFV susceptible(S). Examples of ToBRFV susceptible tomato varieties that can be used as controls are Endeavour F1 and Ramyle F1. As a resistant control a plant deposited as NCIMB 43511 or NCIMB 43512 can be used; a plant of these deposits comprises a modified CCA gene of the invention. NCIMB 43511 comprises a CCA1 gene encoding SEQ ID NO: 8. NCIMB 43512 comprises a CCA1 gene encoding SEQ ID NO: 8. The promoter of the CCA1 gene of NCIMB 43511 and NCIMB 43512 comprises SEQ ID NO: 12.

To determine resistance, seeds of the accessions to be tested are sown in standard seedling trays and seedlings are inoculated 4 weeks after sowing. Inoculum is prepared by grounding leaves of tomato plants that were infected with ToBRFV in a 0.01 M phosphate buffer (pH 7.0) mixed with celite. The seedlings are then dusted with carborundum powder prior to gently rubbing the leaf with inoculum. Resistance is suitably scored on a scale of 0-5; the description of the scales of the scores can be found in Table 2. Observation of the symptoms on the young tomato plants in the bio-assay is preferably done 14-21 days after inoculation (dai).

As used herein, a Solanum lycopersicum plant that is resistant to ToBRFV due to the presence of a modified CCA gene has a score that is 3 or lower than 3, preferably lower than 2.5, when scoring according to Table 2 is used and a bio-assay as described above is performed. In one embodiment, a plant is tolerant to ToBRFV and has a score of 2 or lower than 2, preferably a score of 1 or lower than 1. In another embodiment a plant has field tolerance (FT) to ToBRFV, and has a score of 3 or lower than 3, preferably lower than 2.5, in a bio-assay, but has a score of 2 or lower than 2 in field conditions. As is a criterion in any bio-assay, a representative number of plants has to be scored to obtain a reliable rating, for example 10 plants of a certain line, and the average score should be taken. The susceptible(S) controls in this test should have a score that is higher than 3, preferably higher than 3.5, when the test is performed properly.

A plant of the invention may comprise a modified CCA gene homozygously or heterozygously, i.e. a modified CCA gene can be present on both chromosomes of a chromosome pair in the genome of a plant, or on only one chromosome of a chromosome pair. When two modified CCA genes are present in a certain species, for example in Solanum lycopersicum, they can be present in coupling phase, i.e. two modified CCA genes on the same chromosome, or in repulsion phase, i.e. one modified CCA gene on each complementary chromosome. A plant of the invention may comprise a plant of an inbred line, a hybrid, an open pollinated variety, a doubled haploid, or a plant of a segregating population.

In one embodiment, a plant of the invention is a Solanum lycopersicum plant which may comprise a modified CCA gene as comprised in the genome of a S. lycopersicum plant representative seed of which was deposited with the NCIMB under deposit number 43511 or NCIMB 43512.

In one embodiment, a plant of the invention is a Solanum lycopersicum plant deposited as NCIMB 43511 or NCIMB 43512, or a progeny plant thereof which may comprise one or more or all polymorphisms in the CCA genes that are present in said deposits.

The virus resistance, in particular the ToBRFV resistance, in a plant of the present invention inherits in an intermediate manner. As used herein, intermediate means that a higher level of resistance is found when a modified CCA gene of the invention is homozygously present. The heterozygous presence of a modified CCA gene of the invention however still confers a certain level of ToBRFV resistance. The ToBRFV resistance of both homozygous and heterozygous plants makes the plants more suitable for cultivation under conditions where ToBRFV is present. The improvement on a heterozygous level can also be expressed when the heterozygous plant has two different modified CCA genes, whereby each modified CCA gene comes from a different parent. Therefore both heterozygous and homozygous plants are considered to have improved agronomic characteristics. In addition, heterozygous plants can be used for development of homozygous plants through crossing and selection, which heterozygous plants also therefore form a part of this invention.

The invention further relates to a seed that may comprise a modified CCA gene of the invention, which seed can grow into a plant of the invention. The invention also relates to use of said seed for the production of a plant of the invention, by growing said seed into a plant. The invention also relates to a plant part of a plant of the invention, which may comprise a fruit of a plant of the invention or a seed of a plant of the invention, wherein the plant part may comprise a modified CCA gene in its genome.

The invention further relates to a method for seed production which may comprise growing a plant from a seed of the invention, allowing the plant to produce a fruit with seed, harvesting the fruit, and extracting those seed. Production of the seed is suitably done by selfing or by crossing with another plant that is optionally also a plant of the invention. The seed that is so produced has the capability to grow into a plant that is resistant a positive-strand RNA virus having a TLS, in particular to a virus of the genus Tobamovirus, and more in particular to ToBRFV.

The invention further relates to hybrid seed and to a method for producing said hybrid seed, which may comprise crossing a first parent plant with a second parent plant and harvesting the resultant hybrid seed, wherein the first parent plant and/or the second parent plant is a plant of the invention which may comprise a modified CCA gene of the invention. The resulting hybrid plant that can be grown from the hybrid seed, which may comprise the CCA gene of the invention, which hybrid plant has resistance to a positive-strand RNA virus having a TLS, in particular to a virus of the genus Tobamovirus, and more in particular to ToBRFV, is also a plant of the invention.

The present invention relates to a method for producing a plant that is resistant to a positive-strand RNA virus having a TLS, in particular to a virus of the genus Tobamovirus, and more in particular to ToBRFV, which may comprise introducing a modification in a CCA gene, which modification leads to resistance. Said method may comprise the introduction of a deletion, a substitution, or an insertion in the coding sequence and/or the promoter sequence of a CCA gene. The introduction of such a modification can be done by a mutagenesis approach using a chemical compound, such as ethyl methane sulphonate (EMS); or by using physical means, such as UV-irradiation, fast neutron exposure, or other irradiation techniques.

Introduction of a modification can also be done using a more specific, targeted approach including targeted genome editing by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

Introduction of a modified CCA gene of the invention can also be done through introgression from a plant which may comprise said modified CCA gene, for example from a plant that was deposited as NCIMB 43511 or NCIMB 43512, or from progeny thereof, or from another plant that is resistant to a positive-strand RNA virus having a TLS, in particular to a virus of the genus Tobamovirus, and more in particular to ToBRFV, and in which a modified CCA gene was identified. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied. Progeny of a deposit can be sexual or vegetative descendants of that deposit, which can be selfed and/or crossed, and can be of an F1, F2, or further generation. as long as the descendants of the deposit still comprise a modified CCA gene of that deposit. A plant produced by such method is also a part of the invention.

In one embodiment a modified CCA gene is introgressed into S. lycopersicum from a plant of the species S. pimpinellifolium. In another embodiment a modified CCA gene is introgressed from a S. lycopersicum plant which may comprise the modified CCA gene into a S. lycopersicum plant lacking a modified CCA gene, or into a S. lycopersicum plant which may comprise a different modification in an, optionally different, CCA gene.

Transgenic techniques used for transferring sequences between plants that are sexually incompatible can also be used to produce a plant of the invention, by transferring a modified CCA gene from one species to another. Techniques that can suitably be used comprise general plant transformation techniques known to the skilled person, such as the use of an Agrobacterium-mediated transformation method.

The invention also relates to a method for the production of a plant which is resistant to a positive-strand RNA virus having a TLS, in particular to ToBRFV, said method which may comprise:
a) crossing a plant of the invention which may comprise a modified CCA gene of the invention with another plant;
b) optionally performing one or more rounds of selfing and/or crossing a plant resulting from step a) to obtain a further generation population;
c) selecting from the population resulting from the cross of step a), or from the further generation population of step b), a plant that may comprise a modified CCA gene as defined herein, which plant is resistant against a positive-strand RNA virus having a TLS, in particular to ToBRFV.

The invention also relates to a method for the production of a plant which is resistant to a positive-strand RNA virus having a TLS, in particular to ToBRFV, said method which may comprise:
a) crossing a first parent plant of the invention which may comprise a modified CCA gene of the invention with a second parent plant, which is another plant not comprising a modified CCA gene of the invention, or is another plant that may comprise a different modification in a CCA gene;
b) backcrossing the plant resulting from step a) with the second parent plant for at least three generations;
c) selecting from the third or higher backcross population a plant that may comprise at least the modified CCA gene of the first parent plant of step a).

The invention additionally provides for a method of introducing another desired trait into a plant that is resistant to a positive-strand RNA virus having a TLS, in particular to ToBRFV, which may comprise:
a) crossing a plant which may comprise a modified CCA gene of the invention with a second plant that may comprise the other desired trait to produce F1 progeny;
b) optionally selecting in the F1 for a plant that may comprise the virus resistance and the other desired trait;
c) crossing the optionally selected F1 progeny with one of the parents for at least three generations, to produce backcross progeny;
d) selecting backcross progeny which may comprise the virus resistance and the other desired trait; and
e) optionally repeating steps c) and d) one or more times in succession to produce selected fourth or higher backcross progeny that may comprise the virus resistance and the other desired trait.

Optionally, selfing steps are performed after any of the crossing or backcrossing steps. Selection of a plant which may comprise virus resistance and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and a fruit obtained therefrom.

The invention further relates to a method for the production of a plant which may comprise a modified CCA gene of the invention, which plant is resistant to a positive-strand RNA virus having a TLS, in particular to a Tobamovirus, and more specifically to ToBRFV, by using tissue culture or by using vegetative propagation.

The present invention relates to a method for identification of a plant which may comprise a modified CCA gene of the invention, which plant is resistant to a positive-strand RNA virus having a TLS, in particular to ToBRFV, wherein the identification may comprise determining the presence of a modification in the CCA gene of SEQ ID NO: 1, or in a homologous sequence thereof, and analyzing if the plant which may comprise the modification is resistant to a positive-strand RNA virus having a TLS, in particular to a Tobamovirus, and more specifically to ToBRFV. Determining the presence of a modification in a CCA gene may comprise identification of any of the modifications as described herein, in particular the SNP modifications as presented in Table 4, suitably by using a marker that is designed to identify such modification as its sequence may comprise that particular modification.

The present invention further relates to a method of selection of a plant which is resistant to a positive-strand RNA virus having a TLS, in particular to ToBRFV, the method which may comprise identification of a modified CCA gene of the invention in a plant and subsequently selecting said plant as a plant which is resistant to a positive-strand RNA virus having a TLS, in particular to a Tobamovirus, and more specifically to ToBRFV. Optionally the virus resistance can be confirmed by performing a bio-assay as described in Example 1. The selected plant obtained by such method is also a part of this invention.

The invention also relates to propagation material suitable for producing a plant of the invention, wherein the propagation material is suitable for sexual reproduction, and is in particular selected from a microspore, pollen, an ovary, an ovule, an embryo sac, or an egg cell, or is suitable for vegetative reproduction, and is in particular selected from a cutting, a root, a stem cell, or a protoplast, or is suitable for tissue culture of regenerable cells, and is in particular selected from a leaf, pollen, an embryo, a cotyledon, a hypocotyl, a meristematic cell, a root, a root tip, an anther, a flower, a seed and a stem, and wherein the plant produced from the propagation material may comprise the modified CCA gene of the invention that provides resistance to a positive-strand RNA virus having a TLS, in particular to ToBRFV. A plant of the invention may be used as a source of the propagation material. A tissue culture which may comprise regenerable cells also forms a part of this invention.

The invention further relates to a cell of a plant of the invention. Such a cell may either be in isolated form or a part of the complete plant or parts thereof and still forms a cell of the invention because such a cell may comprise the modified CCA gene of the invention. Each cell of a plant of the invention carries the modified CCA gene of the invention. A cell of the invention may also be a regenerable cell that can regenerate into a new plant of the invention.

The invention further relates to plant tissue of a plant of the invention, which may comprise the modified CCA gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny may comprise the modified CCA gene of the invention. Such progeny can in itself be a plant, a cell, a tissue, or a seed. The progeny can in particular be progeny of a plant of the invention deposited under NCIMB number 43511 or NCIMB 43512. As used herein, progeny may comprise the first and all further descendants from a cross with a plant of the invention, wherein a cross may comprise a cross with itself or a cross with another plant, and wherein a descendant that is determined to be progeny may comprise a modified CCA gene of the invention. Descendants can be obtained through selfing and/or further crossing of the deposit. Progeny also encompasses material that is obtained by vegetative propagation or another form of multiplication.

The invention also relates to a marker for the identification of a modified CCA gene in a plant, which marker may comprise any of the modifications in a CCA gene as described herein and can thereby identify said modifications. A marker of the invention is in particular a marker which may comprise, and thereby suitable for identifying, a SNP modification, i.e. a polymorphism, as presented in Table 4. The use of such marker for identification of a modified CCA gene is also part of this invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Bio-Assay for ToBRFV Resistance in Solanum lycopersicum

S. lycopersicum lines having modifications in one or both CCA genes were observed in a ToBRFV bio-assay. As resistant controls three S. pimpinellifolium sources were included. As susceptible controls Endeavour F1 and Ramyle F1 were used.

Seeds of the accessions to be tested were sown in standard seedling trays and 10 seedlings per accession were inoculated 4 weeks after sowing. Inoculum was prepared by grounding leaves of tomato plants that were infected with ToBRFV in a 0.01 M phosphate buffer (pH 7.0) mixed with celite. Plants were dusted with carborundum powder prior to gently rubbing the leaf with inoculum. Scoring of the symptoms was done according to Table 2 at 19 days after inoculation.

TABLE 2
scoring scales ToBRFV resistance

| score | Symptoms |
|---|---|
| 0 | No symptoms |
| 1 | Not clean, a single spot, some minor discoloration |
| 2 | Mosaic, clear visible symptoms |
| 3 | Severe mosaic, starting deformation in the head |
| 4 | Severe mosaic, necrosis on the stem, serious |
| | deformation in the head, spots in blisters |
| 5 | Dead plant |

Results of the bio-assay are presented in Table 3; the average score of the 10 inoculated seedlings is given. TO313 is a cross between a line with the CCA genotype of NCIMB 43511 and a line with the CCA genotype of NCIMB 43512. A ‘CCA genotype’ means the line has the sequence of CCA1 as in the referred deposit.

TABLE 3
ToBRFV bio-assay scores

| | bio-assay | |
|---|---|---|
| Accession | score | |
| GNL.3951 | 0.3 | resistant control |
| GNL.3919 | 0.5 | resistant control |
| Ramyle F1 | 4.0 | susceptible control |
| Endeavour F1 | 5.0 | susceptible control |
| TO1 | 2.8 | field tolerant line comprising C211R in CCA2 |
| TO310 | 1.7 | CCA genotype of NCIMB 43512 |
| TO311 | 1.3 | CCA genotype of NCIMB 43512 |
| TO313 | 1.5 | CCA genotype of NCIMB 43511 and 43512 |
| TO314 | 1.2 | CCA genotype of NCIMB 43511 |
| TO315 | 1.2 | CCA genotype of NCIMB 43511 |

### Example 2: Identification of Modifications in CCA Genes that Lead to ToBRFV Resistance

Various Solanum lycopersicum populations that segregated for ToBRFV resistance were finemapped to a small region on chromosome 11 that contained only four potential genes which were likely to contribute to the ToBRFV resistance. Whole genome sequences were available in-house for the backgrounds of the resistant and susceptible lines that were used in the development of these populations. Therefore, a SNP-calling approach for the region was done, which means unique polymorphisms in the region were identified through comparing the sequences to each other.

The CCA1 gene was of interest in the analyzed region. CCA1 was found to be a complete CCA gene, which had various polymorphisms between susceptible and resistant material, but all of them led to a protein that harbored the essential domains and active sites of a CCA-adding enzyme.

Different resistant lines were observed to have different polymorphisms. A number of the polymorphisms resulted in non-conservative amino acid changes, which polymorphisms are presented in Table 4.

TABLE 4
Certain SNP modifications correlating with ToBRFV
resistance in S. lycopersicum

| Gene in | | | | | | |
|---|---|---|---|---|---|---|
| S. lycopersium | CCA1 | CCA1 | CCA1 | CCA1 | CCA1 | CCA1 |
| CDS | A948T | C950T | A1348G | A1603G | A1659T | G1737T |
| protein | K316N | A317V | K450E | N535D | R553S | K579N |
| CCA polymorphisms correlating with | T | T | G | G | T | T |
| resistance | | | | | | |
| Polymorphisms present in NCIMB | T | T | A | G | T | T |
| 43511 | | | | | | |
| Polymorphisms present in NCIMB | A | C | A | G | T | T |
| 43512 | | | | | | |
| Wildtype v3 public genome | A | C | A | A | A | G |
| S. lycopersicum (Sl3_00) | | | | | | |

In addition, it was found that the presence of ToBRFV resistance correlated with a deletion in the promoter of the CCA1 gene. In all situations wherein there was a deletion in the promoter, this deletion comprised at least the sequence ATATTTATTT (SEQ ID NO: 4; Table 1), but the deletion could also have several nucleotides more, for example one to ten nucleotides more, in addition to just a deletion of SEQ ID NO: 4. In a certain case it was for example found that the deletion comprised the sequence represented by SEQ ID NO: 18, or the sequence represented by SEQ ID NO: 19, both of which comprise SEQ ID NO: 4.

The deletion in the promoter was present in a TATA rich region, and is therefore believed to be a deletion in the TATA-box of the promoter.

Through analysis of the correlation in segregation of phenotypes and genotypes it was determined that a modification in a CCA gene, which can be a modification in the CCA1 gene, and which can be a modification in the promoter and/or in the coding sequence, was the cause of the ToBRFV resistance of the resistant Solanum lycopersicum plants.

### Example 3: Modification of a CCA Gene to Obtain Resistance to a Positive-Strand RNA Virus Having a TLS

Modifications are introduced in seed of a plant of interest in which resistance to a positive-strand RNA virus having a TLS is needed, for example resistance to a Tobamovirus, such as ToBRFV, ToMV, or TMV. The modification is introduced through mutagenesis, such as an EMS treatment, through radiation means, or through a specific targeted approach, such as CRISPR. When a non-targeted approach such as EMS is used, this is combined with an identification technique such as TILLING. In this way, both for mutagenesis as well as a targeted modification means, a modification in a CCA gene can be generated and identified. The skilled person is familiar with these means for introducing modifications into the genome of a plant of interest.

Modified seed is then germinated and plants are grown, which are crossed or selfed to generate M2 seed. Subsequently a plant screen is performed to identify the modifications in a CCA gene, based on comparison to the wildtype sequence of the one or more CCA genes of that species. For Solanum lycopersicum for example, comparison to SEQ ID NO: 1 or SEQ ID NO: 3 can be done. The skilled person is familiar with TILLING to identify mutations in specific genes (McCallum et. al. (2000) Nature Biotechnology, 18:455-457), and with techniques for identifying nucleotide changes such as DNA sequencing, amongst others.

Plants with a modified CCA gene are homozygous or made homozygous by selfing, crossing, or the use of doubled haploid techniques which are familiar to the skilled person. Plants identified and selected on the basis of a modification in a CCA gene can then be tested for resistance to a positive-strand RNA virus having a TLS, for example resistance to a Tobamovirus, such as ToBRFV, ToMV, or TMV. A plant that is produced, identified and selected in this way is confirmed to have their virus resistance as a result from one or more modifications in the CCA gene.

The invention is further described by the following numbered paragraphs:

1. An agronomically elite tomato (Solanum lycopersicum) plant comprising a modified CCA gene which encodes a CCA-adding enzyme, which modified CCA gene leads to resistance against a positive-strand RNA virus having a TLS, wherein the modified CCA gene is selected from:
a gene comprising a nucleotide sequence that encodes a CCA-adding enzyme according to SEQ ID NO: 8;
a gene comprising a promoter sequence comprising SEQ ID NO: 12;
a gene comprising a nucleotide sequence that encodes a CCA-adding enzyme having a deletion, a substitution, or an insertion of at least one amino acid when compared to SEQ ID NO: 2;
a gene comprising a nucleotide sequence that encodes a CCA-adding enzyme having a deletion, a substitution, or an insertion of at least one amino acid when compared to SEQ ID NO: 8; or
a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution;

2. The agronomically elite plant of paragraph 1, wherein the deletion, substitution, or insertion of at least one amino acid is present in a conserved domain or an active site of the encoded CCA-adding enzyme.

3. The agronomically elite plant of paragraph 1 comprising a modification in the promoter sequence, which promoter sequence comprises SEQ ID NO: 3.

4. The agronomically elite plant of paragraph 3 wherein the modification in a regulatory sequence of the promoter sequence.

5. The agronomically elite plant of paragraph 3, wherein the modification comprises a deletion.

6. The agronomically elite plant of paragraph 1 comprising a combination of two modifications in one CCA gene.

7. The agronomically elite plant of paragraph 6, wherein the modification is in the promoter sequence.

8. The agronomically elite plant of paragraph 6, wherein the modification in the coding sequence.

9. The agronomically elite plant of paragraph 1, wherein the modified CCA gene comprises the gene comprising the nucleotide sequence that encodes the CCA-adding enzyme having at least 90% sequence identity to SEQ ID NO: 8, wherein the CCA-adding enzyme having at least 90% sequence identity to SEQ ID NO: 8 comprises the N535D substitution;
wherein the CCA-adding enzyme having at least 90% sequence identity to SEQ ID NO: 8 further comprises at least one of an R553S substitution or a K579N substitution; or the CCA-adding enzyme having at least 90% sequence identity to SEQ ID NO: 8 comprises any one of those substitutions at a corresponding position of a homologous sequence.

10. The agronomically elite plant of paragraph 9, wherein the CCA-adding enzyme having at least 90% sequence identity to SEQ ID NO: 8 further comprises the R553S substitution and the K579N substitution.

11. The agronomically elite plant of paragraph 1, which is resistant to a positive-strand RNA virus having a TLS.

12. The agronomically elite plant of paragraph 11, wherein the positive-strand RNA virus is a Tobamovirus.

13. The agronomically elite plant of paragraph 12, wherein the Tobamovirus is a Tomato Brown Rugose Fruit Virus (ToBRFV).

14. The agronomically elite plant of paragraph 11, wherein a modified CCA gene is as comprised in the genome of a Solanum lycopersicum plant, representative seed of which was deposited with the NCIMB under deposit number 43511 or NCIMB 43512.

15. A seed of, or from, or that produces the agronomically elite plant of paragraph 1, wherein the agronomically elite plant comprises the modified CCA gene.

16. Propagation material from or suitable for producing the agronomically elite plant of paragraph 1, wherein the propagation material is suitable for sexual reproduction, and comprises a microspore, pollen, ovary, ovule, embryo sac or an egg cell, or the propagation material is suitable for vegetative reproduction and comprises a cutting, root, stem cell, or a protoplast; or the propagation material is suitable for tissue culture of regenerable cells or protoplasts, and comprises a leaf, pollen, embryo, cotyledon, hypocotyl, meristematic cell, root, root tip, anther, flower or a stem; and wherein the propagation material comprises the modified CCA gene.

17. A method for producing the agronomically elite plant of paragraph 1, comprising introducing the modified CCA gene into the genome of the plant.

18. A method for selecting the agronomically elite Solanum lycopersicum plant of paragraph 1, comprising identifying by genetic analysis the modified CCA gene, optionally testing the plant for ToBRFV resistance, and selecting a plant that comprises the modified CCA gene.

19. The method of paragraph 18, wherein the step of identifying further comprises using a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
   or wherein the marker detects a modification on a corresponding position of a homologous sequence having at least 80% sequence identity to SEQ ID NO: 1 or SEQ ID NO: 3.

20. A method for the production of a plant which is resistant to a positive-strand RNA virus comprising a TLS, said method comprising:
a) crossing a first parent plant, with a second parent plant;
b) optionally performing one or more rounds of selfing and/or crossing of the plant resulting from the cross in step a) to obtain a further generation population;
c) selecting from the plant resulting from the cross in step a), or from the further generation population of step b), a plant that comprises a modified CCA gene, wherein the selected plant is resistant to a positive-strand RNA virus comprising a TLS; and
wherein the modified CCA gene is selected from:
   a gene comprising a nucleotide sequence that encodes a CCA-adding enzyme according to SEQ ID NO: 8;
   a gene comprising a promoter sequence comprising SEQ ID NO: 12;
   a gene comprising a nucleotide sequence that encodes a CCA-adding enzyme having a deletion, a substitution, or an insertion of at least one amino acid when compared to SEQ ID NO: 2;
   a gene comprising a nucleotide sequence that encodes a CCA-adding enzyme having a deletion, a substitution, or an insertion of at least one amino acid when compared to SEQ ID NO: 8; or
   a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution.

21. A method for the production of a Solanum lycopersicum plant resistant to ToBRFV, said method comprising:
a) crossing a first parent plant, with a second parent plant;
b) optionally performing one or more rounds of selfing and/or crossing of the plant resulting from the cross in step a) to obtain a further generation population;
c) selecting from the plant resulting from the cross in step a), or from the further generation population of step b), a plant that comprises a modified CCA gene, wherein the selected plant is resistant to ToBRFV; and
wherein the modified CCA gene is selected from:
   a gene comprising a nucleotide sequence that encodes a CCA-adding enzyme according to SEQ ID NO: 8;
   a gene comprising a promoter sequence comprising SEQ ID NO: 12;
   a gene comprising a nucleotide sequence that encodes a CCA-adding enzyme having a deletion, a substitution, or an insertion of at least one amino acid when compared to SEQ ID NO: 2;
   a gene comprising a nucleotide sequence that encodes a CCA-adding enzyme having a deletion, a substitution, or an insertion of at least one amino acid when compared to SEQ ID NO: 8; or
   a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution.

22. The method of paragraph 20, wherein the second parent plant also comprises the modified CCA gene.

23. The method of paragraph 20, wherein the selecting is with a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
   or wherein the marker detects a modification on a corresponding position of a homologous sequence having at least 80% sequence identity to SEQ ID NO: 1 or SEQ ID NO: 3.

24. The method of paragraph 20, wherein a plant which is resistant to ToBRFV is phenotypically selected.

25. The method of paragraph 20, wherein the plant is phenotypically selected with a bio-assay for ToBRFV resistance.

26. The method of paragraph 20, wherein the first parent plant is grown from seed deposited under NCIMB accession number 43511 or NCIMB 43512, or a progeny plant thereof.

27. The method of paragraph 20, wherein the second parent plant also comprises a modified CCA gene.

28. The method of paragraph 21, wherein the selecting is with a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
   or wherein the marker detects a modification on a corresponding position of a homologous sequence having at least 80% sequence identity to SEQ ID NO: 1 or SEQ ID NO: 3.

29. The method of paragraph 21, wherein a plant which is resistant to ToBRFV is phenotypically selected.

30. The method of paragraph 21, wherein the plant is phenotypically selected with a bio-assay for ToBRFV resistance.

31. The method of paragraph 21, wherein the first parent plant is grown from seed deposited under NCIMB accession number 43511 or NCIMB 43512, or a progeny plant thereof.

32. A method for the production of hybrid seed comprising crossing a first parent plant with a second parent plant and harvesting the resultant hybrid seed, wherein the first parent plant and/or the second parent plant is the agronomically elite plant of paragraph 1, and wherein the presence of said modified CCA gene leads to ToBRFV resistance in a plant that is grown from the hybrid seed.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An agronomically elite tomato (Solanum lycopersicum) plant comprising a modified CCA gene which encodes a CCA-adding enzyme, which modified CCA gene leads to resistance against a positive-strand RNA virus having a TLS, wherein the modified CCA gene is selected from:
a gene comprising a nucleotide sequence that encodes a CCA-adding enzyme according to SEQ ID NO: 8;
a gene comprising a promoter sequence comprising SEQ ID NO: 12;
a gene comprising a nucleotide sequence that encodes a CCA-adding enzyme having a deletion, a substitution, or an insertion of at least one amino acid when compared to SEQ ID NO: 2;
a gene comprising a nucleotide sequence that encodes a CCA-adding enzyme having a deletion, a substitution, or an insertion of at least one amino acid when compared to SEQ ID NO: 8; or
a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution.

2. The agronomically elite plant of claim 1, wherein the deletion, substitution, or insertion of at least one amino acid is present in a conserved domain or an active site of the encoded CCA-adding enzyme.

3. The agronomically elite plant of claim 1 comprising a modification in the promoter sequence, which promoter sequence comprises SEQ ID NO: 3.

4. The agronomically elite plant of claim 3 wherein the modification in a regulatory sequence of the promoter sequence.

5. The agronomically elite plant of claim 3, wherein the modification comprises a deletion.

6. The agronomically elite plant of claim 1 comprising a combination of two modifications in one CCA gene.

7. The agronomically elite plant of claim 6, wherein the modification is in the promoter sequence.

8. The agronomically elite plant of claim 6, wherein the modification in the coding sequence.

9. The agronomically elite plant of claim 1, wherein the modified CCA gene comprises the gene comprising the nucleotide sequence that encodes the CCA-adding enzyme having at least 90% sequence identity to SEQ ID NO: 8, wherein the CCA-adding enzyme having at least 90% sequence identity to SEQ ID NO: 8 comprises the N535D substitution;
wherein the CCA-adding enzyme having at least 90% sequence identity to SEQ ID NO: 8 further comprises at least one of an R553S substitution or a K579N substitution; or the CCA-adding enzyme having at least 90% sequence identity to SEQ ID NO: 8 comprises any one of those substitutions at a corresponding position of a homologous sequence.

10. The agronomically elite plant of claim 9, wherein the CCA-adding enzyme having at least 90% sequence identity to SEQ ID NO: 8 further comprises the R553S substitution and the K579N substitution.

11. The agronomically elite plant of claim 1, which is resistant to a positive-strand RNA virus having a TLS.

12. The agronomically elite plant of claim 11, wherein the positive-strand RNA virus is a Tobamovirus.

13. The agronomically elite plant of claim 12, wherein the Tobamovirus is a Tomato Brown Rugose Fruit Virus (ToBRFV).

14. The agronomically elite plant of claim 11, wherein a modified CCA gene is as comprised in the genome of a Solanum lycopersicum plant, representative seed of which was deposited with the NCIMB under deposit number 43511 or NCIMB 43512.

15. A seed of, or from, or that produces the agronomically elite plant of claim 1, wherein the agronomically elite plant comprises the modified CCA gene.

16. Propagation material from or suitable for producing the agronomically elite plant of claim 1, wherein the propagation material is suitable for sexual reproduction, and comprises a microspore, pollen, ovary, ovule, embryo sac or an egg cell, or the propagation material is suitable for vegetative reproduction and comprises a cutting, root, stem cell, or a protoplast; or the propagation material is suitable for tissue culture of regenerable cells or protoplasts, and comprises a leaf, pollen, embryo, cotyledon, hypocotyl, meristematic cell, root, root tip, anther, flower or a stem; and wherein the propagation material comprises the modified CCA gene.

17. A method for producing the agronomically elite plant of claim 1, comprising introducing the modified CCA gene into the genome of the plant.

18. A method for selecting the agronomically elite Solanum lycopersicum plant of claim 1, comprising identifying by genetic analysis the modified CCA gene, optionally testing the plant for ToBRFV resistance, and selecting a plant that comprises the modified CCA gene.

19. The method of claim 18, wherein the step of identifying further comprises using a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
or wherein the marker detects a modification on a corresponding position of a homologous sequence having at least 80% sequence identity to SEQ ID NO: 1 or SEQ ID NO: 3.

20. A method for the production of a plant which is resistant to a positive-strand RNA virus comprising a TLS, said method comprising:
a) crossing a first parent plant, with a second parent plant;
b) optionally performing one or more rounds of selfing and/or crossing of the plant resulting from the cross in step a) to obtain a further generation population;
c) selecting from the plant resulting from the cross in step a), or from the further generation population of step b), a plant that comprises a modified CCA gene, wherein the selected plant is resistant to a positive-strand RNA virus comprising a TLS; and
wherein the modified CCA gene is selected from:
a gene comprising a nucleotide sequence that encodes a CCA-adding enzyme according to SEQ ID NO: 8;
a gene comprising a promoter sequence comprising SEQ ID NO: 12;
a gene comprising a nucleotide sequence that encodes a CCA-adding enzyme having a deletion, a substitution, or an insertion of at least one amino acid when compared to SEQ ID NO: 2;
a gene comprising a nucleotide sequence that encodes a CCA-adding enzyme having a deletion, a substitution, or an insertion of at least one amino acid when compared to SEQ ID NO: 8; or
a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution.

21. A method for the production of a Solanum lycopersicum plant resistant to ToBRFV, said method comprising:
a) crossing a first parent plant, with a second parent plant;
b) optionally performing one or more rounds of selfing and/or crossing of the plant resulting from the cross in step a) to obtain a further generation population;
c) selecting from the plant resulting from the cross in step a), or from the further generation population of step b), a plant that comprises a modified CCA gene, wherein the selected plant is resistant to ToBRFV; and
wherein the modified CCA gene is selected from:
a gene comprising a nucleotide sequence that encodes a CCA-adding enzyme according to SEQ ID NO: 8;
a gene comprising a promoter sequence comprising SEQ ID NO: 12;
a gene comprising a nucleotide sequence that encodes a CCA-adding enzyme having a deletion, a substitution, or an insertion of at least one amino acid when compared to SEQ ID NO: 2;
a gene comprising a nucleotide sequence that encodes a CCA-adding enzyme having a deletion, a substitution, or an insertion of at least one amino acid when compared to SEQ ID NO: 8; or
a gene comprising a nucleotide sequence that encodes a CCA-adding enzyme having at least 90% sequence identity to SEQ ID NO: 8, wherein the CCA-adding enzyme having at least 90% sequence identity to SEQ ID NO: 8 comprises a N535D substitution.

22. The method of claim 20, wherein the second parent plant also comprises the modified CCA gene.

23. The method of claim 20, wherein the selecting is with a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
or wherein the marker detects a modification on a corresponding position of a homologous sequence having at least 80% sequence identity to SEQ ID NO: 1 or SEQ ID NO: 3.

24. The method of claim 20, wherein a plant which is resistant to ToBRFV is phenotypically selected.

25. The method of claim 20, wherein the plant is phenotypically selected with a bio-assay for ToBRFV resistance.

26. The method of claim 20, wherein the first parent plant is grown from seed deposited under NCIMB accession number 43511 or NCIMB 43512, or a progeny plant thereof.

27. The method of claim 20, wherein the second parent plant also comprises a modified CCA gene.

28. The method of claim 21, wherein the selecting is with a marker that detects a modification selected from the group consisting of:
an A to T SNP on position 948 of SEQ ID NO: 1,
a C to T SNP on position 950 of SEQ ID NO: 1,
an A to G SNP on position 1348 of SEQ ID NO: 1,
an A to G SNP on position 1603 of SEQ ID NO: 1,
an A to T SNP on position 1659 of SEQ ID NO: 1,
a G to T SNP on position 1737 of SEQ ID NO: 1, and
a deletion in SEQ ID NO: 3 comprising SEQ ID NO: 4,
or wherein the marker detects a modification on a corresponding position of a homologous sequence having at least 80% sequence identity to SEQ ID NO: 1 or SEQ ID NO: 3.

29. The method of claim 21, wherein a plant which is resistant to ToBRFV is phenotypically selected.

30. The method of claim 21, wherein the plant is phenotypically selected with a bio-assay for ToBRFV resistance.

31. The method of claim 21, wherein the first parent plant is grown from seed deposited under NCIMB accession number 43511 or NCIMB 43512, or a progeny plant thereof.

32. A method for the production of hybrid seed comprising crossing a first parent plant with a second parent plant and harvesting the resultant hybrid seed, wherein the first parent plant and/or the second parent plant is the agronomically elite plant of claim 1, and wherein the presence of said modified CCA gene leads to ToBRFV resistance in a plant that is grown from the hybrid seed.
